(19) United States
(12) Patent Application Publication
(10) Pub. No.: US 2015/0190413 A1
(43) Pub. Date: Jul. 9, 2015

(54) COMPOSITIONS AND METHODS FOR THE TREATMENT OF BLADDER CANCER

(71) Applicant: Endo Pharmaceuticals Solutions Inc., Malvern, PA (US)

(72) Inventors: John CHABER, Westford, MA (US); Petr KUZMA, Princeton, NJ (US); Agis KYDONIEUS, Kendall Park, NJ (US)

(73) Assignee: Endo Pharmaceuticals Solutions Inc., Malvern, PA (US)

(21) Appl. No.: 14/663,830

(22) Filed: Mar. 20, 2015

Related U.S. Application Data
(63) Continuation of application No. 12/324,019, filed on Nov. 26, 2008, now abandoned.
(60) Provisional application No. 60/991,596, filed on Nov. 30, 2007.

(51) Int. Cl. A61K 31/704; A61K 47/20; A61K 9/127; A61K 47/10; A61K 47/40; A61K 47/34; A61K 47/32; A61K 47/26; A61K 9/00; A61K 45/06

(52) U.S. Cl. CPC A61K 31/704; A61K 9/0034; A61K 47/20; A61K 9/127; A61K 45/06; A61K 47/40; A61K 47/34; A61K 47/32; A61K 47/26; A61K 47/10

(57) ABSTRACT

Compositions and methods for the treatment of bladder cancer include intravesical dosage forms of a neoplastic agent and a permeation enhancer. The neoplastic agent may be valrubicin. Pharmaceutical compositions include intravesical dosage forms of a neoplastic agent complexed liposomes. Tight junction openers may be used for the effective delivery of the neoplastic agent.

### CROSS-REFERENCE TO RELATED APPLICATIONS

This application claims the benefit of U.S. Provisional Patent Application Nos. 60/991,596, filed Nov. 30, 2007, the entire contents of which are incorporated herein by reference for any and all purposes.

### FIELD

The present invention relates generally to the field of cancer therapy. In particular, therapies are provided for cancers developed in a hollow body structure of a patient, such as the bladder, colon, mouth and stomach.

### BACKGROUND

The following description is provided to assist the understanding of the reader. None of the information provided or references cited is admitted to be prior art to the present invention.

Neoplasms of the bladder generally originate as pre-malignant lesions and can develop into invasive cancer. Some will go on to metastatic growth. The most common bladder neoplasm is a transitional cell carcinoma of epithelial origin. Patients with superficial bladder malignancy have a good prognosis but deep invasion of the underlying musculature reduces five year survival to about 50%.

Surgery is the main treatment method. The extent of surgery is dependent on the pathological stage of the disease. Early disease is generally treated by intravesical chemotherapy and transurethral resection. Locally invasive disease can usually be managed only by radical cystectomy and urinary diversion. Surgery is often combined with adjuvant intravesicular installation of chemotherapeutic or immunotherapeutic agents to reduce the incidence and severity of recurrence of cancer either at the same site or at another site on the bladder wall. Definitive (curative) radiotherapy is generally reserved for bladder cancer patients who are not candidates for surgery. For superficial, low-grade disease, chemotherapy is applied intravesically (directly into the bladder) to concentrate the drug at the tumor site and eliminate any residual tumor mass after resection. Systemic chemotherapy can also be used to manage advanced bladder cancer.

One such chemotherapy agent used in bladder cancer is Valstar®. Valstar® is a formulation of valrubicin in ethanol that is instilled into bladders to treat bladder cancers. It may be used instead of, or after, transurethral resection of the bladder to target cancer cells. However, it is known that such formulations are irritating to some patients and the formulations are voided from the bladder before full efficacy is achieved. Thus, vehicles for administration of valrubicin are needed to reduce the irritation and increase the efficacy of the treatment.

### SUMMARY

In one aspect, compositions and methods for the treatment of bladder cancer comprise intravesical dosage forms of a neoplastic agent. In another aspect, a pharmaceutical composition is provided including an effective amount of a neoplastic agent and dimethyl sulfoxide in an intravesical dosage form. In some embodiments, the effective amount of valrubicin is from about 5 mg/mL to about 100 mg/mL, from about 10 mg/mL to about 90 mg/mL, from about 15 mg/mL to about 80 mg/mL, from about 20 mg/mL to about 70 mg/mL, from about 25 mg/mL to about 70 mg/mL, from about 30 mg/mL to about 60 mg/mL, from about 35 mg/mL to about 50 mg/mL, or from about 35 mg/mL to about 45 mg/mL. In some embodiments, the pharmaceutical composition includes one or more additional chemical permeation enhancers selected from ethanol, isopropanol, dimethylacetamide, dimethylformamide, decylmethylsulfoxide, 2-pyrrolidone, N-ethyl-2-pyrrolidone, capric acid, linoleic acid, ureas, sodium dodecyl sulfate, sodium lauryl sulfate, and mixtures of any two or more thereof. In other embodiments, the effective amount of valrubicin and dimethyl sulfoxide is sufficient to treat bladder cancer.

In some embodiments, the pharmaceutical compositions include a junction opener. In some embodiments, the junction opener may be trimethyl-chitosan, mono-N-carboxymethyl chitosan, N-diethyl methyl chitosan, sodium caprate, cytochalasin B, IL-1, polycarbophil, carbopol 934P, N-sulfato-N,O-carboxymethylchitosan, Zounla occludens toxin, 1-palmitoyl-2-glutaroyl-sn-glycero-3-phosphocholine, or a mixture of any two or more thereof. The junction opener may be present in the formulation from about 1 to about 15 percent by weight/volume of the dosage form.

In some embodiments, the pharmaceutical compositions include a polyethoxylated castor oil. The polyethoxylated castor oil may be Cremophor, according to other embodiments. In some embodiments, the Cremophor and dimethyl sulfoxide are provided in equal amounts. In some embodiments, the pharmaceutical compositions include a junction opener. The junction opener may be trimethyl-chitosan, mono-N-carboxymethyl chitosan, N-diethyl methyl chitosan, sodium caprate, cytochalasin B, IL-1, polycarbophil, carbopol 934P, N-sulfato-N,O-carboxymethylchitosan, Zounla occludens toxin, 1-palmitoyl-2-glutaroyl-sn-glycero-3-phosphocholine, or a mixture of any two or more thereof.

In some embodiments, the pharmaceutical compositions include a mucin-degrading compound. In some embodiments, the mucin-degrading compound is selected from the group consisting of: trypsin, hyaluronidase, protamine sulfate, and norepinephrine.

In some embodiments, the pharmaceutical compositions include a bioadhesive or mucoadhesive agent. In some embodiments, the mucoadhesive agent is polyacrylic acid. In some embodiments, the pharmaceutical composition further includes an ionic or non-ionic surfactant, a polyvinyl pyrrolidone, alginates, a polyacrylic acid, or a mixture of any two or more thereof. Exemplary ionic and non-ionic surfactants include polyoxyethylene castor oil derivatives, block copolymers of ethylene oxide and propylene oxide, sorbitan fatty acid esters, or a mixture of any two or more thereof. Exemplary polyacrylic acids include Carbomer 934P, Carbomer 940, Carbomer 941, Carbomer 974P, Carbomer 980, Carbomer 1342, polycarbophil, calcium polycarbophil, or a mixture of any two or more thereof.

In another aspect, a pharmaceutical composition is provided including an effective amount of valrubicin and 2-hydroxy-propyl-β-cyclodextran in an intravesical dosage fog in. In some embodiments, the amount of 2-hydroxy-propyl-β-cyclodextran is from about 1 to about 5 percent weight/volume of the dosage form. In some embodiments, the pharmaceutical composition also includes a tight junction opener. In some embodiments, the junction opener is trimethyl-chitosan, mono-N-carboxymethyl chitosan, N-diethyl methyl chitosan, sodium caprate, cytochalasin B, IL-1, polycarbophil, carbopol 934P, N-sulfato-N,O-carboxymethylchitosan, Zounla occludens toxin, 1-palmitoyl-2-glutaroyl-sn-glycero-3-phosphocholine, or a mixture of any two or more thereof. In some embodiments, the pharmaceutical compositions also include a bioadhesive or mucoadhesive agent. In some embodiments, the mucoadhesive agent is polyacrylic acid.

In another aspect, a pharmaceutical composition is provided including a liposomal dosage form comprising an effective amount of liposome-entrapped valrubicin, where, the liposome comprises at least one liposome forming material selected from the group consisting of: phosphatidyl choline and phosphatidyl ethanolamine. In some embodiments, the liposome forming material comprises from about 4 to about 8 percent by weight phosphatidyl choline. In other embodiments, the pharmaceutical composition includes from about 0.5 to about 2 percent by weight cholesterol. In some embodiments, the pharmaceutical composition includes from about 1 to about 6 percent by weight of one or more sphingolipids that are D-glucosyl-β1-1′ceramide (C8); D-glucosyl-β1-1′ceramide (C12); D-glucosyl-β1,1′ N-palmitoyl-D-erythro-sphinosine; D-galactosyl-β1-1′ceramide (C8); D-galactosyl-β1-1′ceramide (12); D-galactosyl-β1-1′-N-Nervonyl-D-erythro-sphingosine; or D-glactose-β1-1′ ceramide (C8); and D-glactose-β1-1′ ceramide (C12). In some embodiments, the liposome forming material comprises from about 2 to about 8 percent by weight phosphatidyl ethanolamine. In other embodiments, the pharmaceutical composition includes from about 1 to about 5 percent by weight phosphatidyl inositol. In other embodiments, the pharmaceutical composition includes from about 0.5 to about 1 percent by weight oleic acid. In other embodiments, the pharmaceutical composition includes from about 0.5 to about 2 percent by weight cholesterol. In other embodiments, the pharmaceutical composition includes from about 3 to about 4 percent by weight diglyceride-succinate. In some embodiments, the pharmaceutical composition includes an oil. Such oils may include, but are not limited to safflower, triacetin, and cottonseed. In some embodiments, the pharmaceutical composition includes a permeation enhancer. In other embodiments, the permeation enhancer is oleic acid, capric acid, linoleic acid, ureas, sodium dodecyl sulfate, sodium lauryl sulfate, or a mixture of any two or more thereof.

In another aspect, a pharmaceutical composition is provided including an effective amount of emulsion-entrapped valrubicin; wherein the emulsion includes at least one emulsion-forming material selected from phosphatidyl choline, phosphatidyl ethanolamine and oil. In some embodiments, the oil is selected from the group consisting of: safflower, triacetin, and cottonseed. In other embodiments, the pharmaceutical composition further includes a permeation enhancer. In some embodiments, the permeation enhancer is dimethyl sufoxide, oleic acid, capric acid, linoleic acid, ureas, sodium dodecyl sulfate, sodium lauryl sulfate, or a mixture of any two or more thereof.

In another aspect, a method for treating bladder cancer is provided including administering a composition comprising an effective amount of valrubicin and dimethyl sulfoxide. In some embodiments, the composition is administered intravesically after transurethral resection of the bladder.

In another aspect, a method for treating bladder cancer is provided including administering a liposomal dosage form including an effective amount of liposome-entrapped valrubicin, wherein the liposome includes at least one liposome forming material selected from phosphatidyl choline and phosphatidyl ethanolamine.

In another aspect, a method for treating bladder cancer is provided including administering an emulsion dosage form including an effective amount of emulsion-entrapped valrubicin; wherein the emulsion includes at least one emulsion-forming material selected from phosphatidyl choline, phosphatidyl ethanolamine and oil. In some embodiments, the oil is selected from the group consisting of: safflower, triacetin, and cottonseed. In other embodiments, the dosage form further includes a permeation enhancer. In some embodiments, the permeation enhancer is dimethyl sulfoxide, oleic acid, capric acid, linoleic acid, ureas, sodium dodecyl sulfate, sodium lauryl sulfate, or a mixture of any two or more thereof.

### BRIEF DESCRIPTION OF THE DRAWINGS

FIG. 1 is a graph comparing the mean inflammation score of a negative control saline formulation, a positive control Valstar formulation, and a valrubicin/DMSO formulation, according to one embodiment.

FIG. 2 is a graph comparing the mean inflammation scores of a Valstar formulation, a valrubicin/DMSO formulation, and a valrubicin/liposomal formulation, according to some embodiments.

FIG. 3 is a graph comparing the mean inflammation scores of Formulations 4, 9, 11, and 12, according to some embodiments.

### DETAILED DESCRIPTION

Before the present compositions and methods are described, it is to be understood that they are not limited to the particular process, composition, or methodology described, as these may vary. It is also to be understood that the terminology used in the description is for the purpose of describing the particular versions or embodiments only, and is not intended to limiting. Unless defined otherwise, all technical and scientific terms used herein have the same meanings as commonly understood by one of ordinary skill in the art. All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure. Nothing herein is to be construed as an admission that the invention is not entitled to antedate such disclosure by virtue of prior invention.

Compounds described herein may contain an asymmetric center and may thus exist as enantiomers. Where the compounds possess two or more asymmetric centers, they may additionally exist as diastereomers. The compounds include all such possible stereoisomers as substantially pure resolved enantiomers, racemic mixtures thereof, as well as mixtures of diastereomers. The formulas are shown without a definitive stereochemistry at certain positions. The compounds include all stereoisomers of such formulas and pharmaceutically acceptable salts thereof. Diastereoisomeric pairs of enantiomers may be separated by, for example, fractional crystallization from a suitable solvent, and the pair of enantiomers thus obtained may be separated into individual stereoisomers by conventional means, for example by the use of an optically active acid or base as a resolving agent or on a chiral HPLC column. Further, any enantiomer or diastereomer of a compound of the general formula may be obtained by stereospecific synthesis using optically pure starting materials or reagents of known configuration.

In the description that follows, a number of terms are utilized extensively. Definitions are herein provided to facilitate understanding of the various embodiments. The terms defined below are more fully defined by reference to the specification as a whole. Units, prefixes, and symbols may be denoted in their accepted SI form.

As used herein, the term “about” means plus or minus 10% of the numerical value of the number with which it is being used.

As used herein, the term “administration” or “administering” when used in conjunction with a therapeutic means to administer a therapeutic directly into or onto a target tissue or to administer a therapeutic to a patient whereby the therapeutic positively impacts the tissue to which it is targeted. Thus, as used herein, the term “administering”, when used in conjunction with a neoplastic agent, can include, but is not limited to, providing a neoplastic agent into or onto the target tissue, or providing a neoplastic agent to a subject by, e.g., intravesical administration.

As used herein, the term “controlled release” refers to a formulation or device designed to consistently release a predetermined, therapeutically effective amount of drug or other active agent such as a neoplastic agent over an extended period of time, with the result being a reduction in the number of treatments necessary to achieve the desired therapeutic effect. As such, a controlled release formulation would decrease the number of treatments necessary to achieve the desired effect in terms of treating cancer or preventing cancer recurrence. The controlled release formulations achieve a desired pharmacokinetic profile in a subject, preferably commencement of the release of the active agent substantially immediately after placement in a delivery environment, followed by consistent, sustained, preferably zero-order or near zero-order release of the active agent. Controlled release includes the predetermined, consistent release of active agent from the dosage formulation at a rate such that a therapeutically beneficial level of the active agent is maintained over an extended period of at about one day to about one week, one week to about one month, or about one month to about two months.

The term “inhibiting” includes the administration of a compound to prevent the onset of the symptoms, alleviating the symptoms, or eliminating the disease, condition or disorder.

The terms “patient” and “subject” mean all animals including humans. Examples of patients or subjects include humans, cows, dogs, cats, goats, sheep, and pigs.

By “pharmaceutically acceptable”, it is meant the carrier, diluent or excipient must be compatible with the other ingredients of the formulation and not deleterious to the recipient thereof.

The term “pharmaceutically acceptable salts, esters, amides, and prodrugs” as used herein refers to those carboxylate salts, amino acid addition salts, esters, amides, and prodrugs of the compounds which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds.

The term “prodrug” refers to compounds that are rapidly transformed in vivo to yield the parent compounds of the above formula, for example, by hydrolysis in blood. A thorough discussion is provided in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are incorporated herein by reference.

In addition, the compounds can exist in unsolvated as well as solvated forms with pharmaceutically acceptable solvents such as water, ethanol, and the like. In general, the solvated forms are considered equivalent to the unsolvated forms.

The term “salts” refers to the relatively non-toxic, inorganic and organic acid addition salts of compounds. These salts can be prepared in situ during the final isolation and purification of the compounds or by separately reacting the purified compound in its free base form with a suitable organic or inorganic acid and isolating the salt thus formed. Representative salts include the acetate, hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate and laurylsulphonate salts, and the like. These may include cations based on the alkali and alkaline earth metals, such as sodium, lithium, potassium, calcium, magnesium, and the like, as well as non-toxic ammonium, tetramethylammonium, tetraethyl ammonium, methylamine, dimethylamine, trimethylamine, triethylamine, ethylamine, and the like. (See, for example, S. M. Berge et al., “Pharmaceutical Salts,” J. Pharm. Sci., 1977, 66:1-19 which is incorporated herein by reference.).

As used herein, the term “therapeutic” means an agent utilized to treat, combat, ameliorate, prevent or improve an unwanted condition or disease of a patient. In part, embodiments are directed to the treatment of bladder cancer or the decrease in the recurrence in bladder cancer compared to subjects not administered the therapeutic.

A “therapeutically effective amount” or “effective amount” of a composition is a predetermined amount calculated to achieve the desired effect, i.e., to decrease or prevent bladder cancer or the recurrence of bladder cancer. The activity contemplated includes both medical therapeutic and/or prophylactic treatment, as appropriate. The specific dose of a compound administered to obtain therapeutic and/or prophylactic effects will, of course, be determined by the particular circumstances surrounding the case, including, for example, the compound administered, the route of administration, and the condition being treated. However, it will be understood that the effective amount administered will be determined by the physician in the light of the relevant circumstances including the condition to be treated, the choice of compound to be administered, and the chosen route of administration, and therefore the above dosage ranges are not intended to limit the scope in any way. A therapeutically effective amount of compound is typically an amount such that when it is administered in a physiologically tolerable excipient composition, it is sufficient to achieve an effective systemic concentration or local concentration in the tissue.

The terms “treat,” “treated,” or “treating” as used herein refers to both therapeutic treatment and prophylactic or preventative measures, wherein the object is to prevent or slow down (lessen) an undesired physiological condition, disorder or disease, or to obtain beneficial or desired clinical results. Beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

### Compositions and Methods

Pharmaceutical compositions are provided which have activity as anti-cancer agents and to methods for the treatment of bladder cancer in patients. In one aspect, pharmaceutical compositions comprise a neoplastic agent (NA) and a permeation enhancer. In one embodiment, composition comprises an effective amount of the valrubicin and the permeation enhancer dimethyl sulfoxide (DMSO). In other embodiments, the composition comprises an effective amount of valrubicin, the permeation enhancer DMSO, and an additive.

Methods are also provided to overcome a series of barriers that prevent effective delivery of a neoplastic agent to the bladder wall. In particular, barriers to effective delivery include (a) the mucin layer that surrounds the bladder wall, (b) the short time interval that the neoplastic agent is able to stay in contact with the wall, and (c) permeation of the neoplastic agent through the bladder wall. The compositions and methods adequately treat the cancer cells that may have invaded the underlying musculature.

In various embodiments, the neoplastic agent or chemotherapeutic agent includes the anti-proliferative agents mitomycin C, valrubicin, and doxorubicin, taxol, and BCG. In a preferred embodiment, the neoplastic agent is valrubicin. Valrubicin (N-trifluoroacetyladriamycin-14-valerate, Valstar®) is a chemotherapy drug used to treat bladder cancer. Valrubicin is a semisynthetic analog of the anthracycline doxorubicin, and is administered by infusion directly into the bladder.

In one embodiment, the pharmaceutical composition comprises a neoplastic agent and an acceptable chemical skin permeation enhancer. Chemical permeation enhancers disrupt the ordered structure of the intercellular lipid bilayers (lipophilic pathway) as well as the intracellular environment (hydrophilic pathway). There are many families of chemical enhancers including alcohols (ethanol, isopropanol), amines and amides (dimethylacetamide, dimethylformamide), sulfoxides (decylmethylsulfoxide, dimethylsulfoxide (DMSO)), pyrrolidones (2-pyrrolidone, N-ethyl-2-pyrrolidone), fatty acids (capric acid, linoleic acid), ureas and unsaturated cyclic ureas, surfactants (sodium dodecyl sulfate, sodium lauryl sulfate) and others (see Percutaneous Permeation Enhancers, CRC Press, 1995).

In particular embodiments, the chemical permeation enhancer is compatible with valrubicin. In a specific embodiment, DMSO is an acceptable chemical skin permeation enhancer. DMSO is a preferred skin permeation enhancer because (a) it has been approved for use in instillation into the bladder (Rimso 50, PDR, 58^{th} Edition, 2004, p. 1215), and (b) it may reduce discomfort associated with the rapidly volatilizing ethanol in currently available formulations. Furthermore, DMSO will carry some valrubicin into the underlining musculature, without affecting the amount reaching the systemic circulation. Due to the hydrophilic nature of the bladder tissues, valrubicin will precipitate upon contact. Accordingly, formulations comprising valrubicin and DMSO are expected to kill cancer cells that have invaded the underlying muscle.

As noted above, the composition may also contain an additive in addition to the valrubicin and DMSO. In some embodiments, such additives include both ionic and non-ionic surfactants such as polyoxyethylene castor oil derivatives, block copolymers of ethylene oxide and propylene oxide, sorbitan fatty acid esters; polyvinyl pyrrolidone; alginates; and polyacrylic acids.

Polyoxyethylene castor oil derivatives include, but are not limited to polyoxyethyleneglyceroltriricinoleate or polyoxyl 35 castor oil (Cremophor®EL, BASF Corp.), polyoxyethyleneglycerol oxystearate (Cremophor®RH 40 (polyethyleneglycol 40 hydrogenated castor oil), and Cremophor®RH 60 (polyethyleneglycol 60 hydrogenated castor oil), BASF Corp). Block copolymers of ethylene oxide and propylene oxide include, but are not limited to, polyoxyethylene polyoxypropylene block copolymers or polyoxyethylenepolypropylene glycol, such as Poloxamer®124, Poloxamer® 188, Poloxamer®237, Poloxamer®388, Poloxamer® 407 (BASF Wyandotte Corp.), and the like. Sorbitan fatty acid esters include, but are not limited to mono fatty acid esters of polyoxyethylene (20) sorbitan, for example, polyoxyethylene (20) sorbitan monooleate (Tween®80, aka Polysorbate®80), polyoxyethylene (20) sorbitan monostearate (Tween®60), polyoxyethylene (20) sorbitan monopalmitate (Tween®40), polyoxyethylene (20) sorbitan monolaurate (Tween®20), and the like. Polyacrylic acids may be alternatively known as Carbomer 934P, 940, 941, 974P, 980, 1342, polycarbophil, and calcium polycarbophil (BF Goodrich).

DMSO has been used to enhance the penetration of agents into the bladder wall, however, the state of the art is such that, prior to the present application, it was believed that DMSO administration resulted in cell death or fixation of the cells, which can reduce the efficacy of any chemotherapeutic treatment being administered via the DMSO. For example, Borzelleca et al. (Investigative Urology 6(1), 43-52 (1968)) describes the use of DMSO for the administration of sodium salicylate to the bladders of rabbits. However, Borzelleca showed that the epithelium of the bladder is sensitive to even five percent solutions of DMSO in water, with severe reactions such as loss of epithelial cells at twenty percent solutions of DMSO in water. Id. At one hundred percent DMSO, the cells, while appearing normal, are fixed, as if a histological fixative were applied to the cells. Id. Thus, at the time, it was expected that DMSO would produce effects adverse to those effects that were desired.

In one embodiment, the pharmaceutical composition includes a neoplastic agent and an enzyme or compound that degrades the mucin layer coating the bladder wall. The mucin layer coating the bladder wall is composed of glycosaminoglycans, hyaluronic acid and chondroitin sulfate which are elevated in bladder cancer patients. While not wishing to be limited to any particular mechanism, it is predicted that if the mucin layer is removed, the chemotherapeutic agent can reach the lumina layer of the bladder wall and become more effective in treating the disease. Enzymes as well as other compounds can degrade the mucin layer. Examples include trypsin and animal-sourced and recombinant hyaluronidase enzymes. Protamine sulfate and norepinephrine are other compounds that can also be used.

In one embodiment, the pharmaceutical composition comprises a neoplastic agent and a bioadhesive or mucoadhesive that will form at least a monomolecular layer of the formulation on the walls of the bladder for an extended period of time. Bioadhesives are used to promote dosage form residence time as well as improve intimacy of contact with various absorptive membranes, such as the mucosal tissue of the bladder wall. Besides acting as platforms for controlled release, bioadhesive polymers can themselves exert some control over the rate and amount of drug release and thus contribute to the therapeutic advantage of such systems (Bioadhesive Drug Delivery Systems, CRC Press, p. 66 (1990)). Representative natural polymers include proteins such as zein, modified zein, casein, gelatin, gluten, serum albumin, and collagen, polysaccharides such as cellulose, dextrans, and polyhyaluronic acid. Representative synthetic polymers include polyphosphazenes, poly(vinyl alcohols), polyamides, polycarbonates, polyacrylates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes and copolymers thereof. Examples of suitable polyacrylates include poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate) and poly(octadecyl acrylate).

The polymers described above can be separately characterized as biodegradable, non-biodegradable, and bioadhesive polymers, as discussed in more detail below. Representative synthetic degradable polymers include polyhydroxy acids such as polylactides, polyglycolides and copolymers thereof, poly(ethylene terephthalate), poly(butic acid), poly(valeric acid), poly(lactide-co-caprolactone), polyanhydrides, polyorthoesters and blends and copolymers thereof. Representative natural biodegradable polymers include polysaccharides such as alginate, dextran, cellulose, collagen, and chemical derivatives thereof (substitutions, additions of chemical groups, for example, alkyl, alkylene, hydroxylations, oxidations, and other modifications routinely made by those skilled in the art), and proteins such as albumin, zein and copolymers and blends thereof, alone or in combination with synthetic polymers. In general, these materials degrade either by enzymatic hydrolysis or exposure to water in vivo, by surface or bulk erosion. Examples of non-biodegradable polymers include ethylene vinyl acetate, poly(meth)acrylic acid, polyamides, polyethylene, polypropylene, polystyrene, polyvinyl chloride, polyvinylphenol, and copolymers and mixtures thereof. Hydrophilic polymers and hydrogels tend to have bioadhesive properties. Hydrophilic polymers that contain carboxylic groups (e.g., poly[acrylic acid]) tend to exhibit the best bioadhesive properties. Polymers with the highest concentrations of carboxylic groups are preferred when bioadhesiveness on soft tissues is desired. Various cellulose derivatives, such as sodium alginate, carboxymethylcellulose, hydroxymethylcellulose and methylcellulose also have bioadhesive properties. Some of these bioadhesive materials are water-soluble, while others are hydrogels. Polymers such as hydroxypropylmethylcellulose acetate succinate (HPMCAS), cellulose acetate trimellitate (CAT), cellulose acetate phthalate (CAP), hydroxypropylcellulose acetate phthalate (HPCAP), hydroxypropylmethylcellulose acetate phthalate (HPMCAP), and methylcellulose acetate phthalate (MCAP) may be utilized to enhance the bioavailability of drugs with which they are complexed. Rapidly bioerodable polymers such as poly(lactide-co-glycolide), polyanhydrides, and polyorthoesters, whose carboxylic groups are exposed on the external surface as their smooth surface erodes, can also be used as bioadhesives for delivery of neoplastic agents.

In one embodiment, the pharmaceutical composition comprises a neoplastic agent and one or more tight junction opening compounds to allow the neoplastic agent to penetrate into the underlying musculature. Tight junction opening compounds regulate paracellular drug transport, affording transient, rapid and reversible tight junction permeability in epithelial tissue. One example of those modifiers is 1-palmitoyl-2-glutaroyl-sy-glycero-3-phosphocholine (Nastech Pharmaceutical). Others examples include N-diethyl methyl chitosan (International Journal of Pharmaceutics 293:83, 2005); sodium caprate and cytochalasin B (Digestive Diseases and Sciences 43: 1547, 1998); IL-1 (J. Immunology 178:4641, 2007); polycarbophil, carbopol 934P, carbomers and trimethyl chitosan (Biomaterials 23 (1): 153, 2002 and Pharm. Res 18 (11):1638, 2001); mono-carboxylated chitosan (Adv. Drug Delivery Reviews 52 (2):117, 2001); N-sulfato-N,O-carboxymethylchitosan (U.S. Pat. No. 7,265,097); and Zounla occludens toxin and fragments (Adv. Drug Delivery Reviews 58:15, 2006). Accordingly, in some embodiments, tight junction modulators in conjunction with chemical enhancers and other excipients affecting the three barriers mentioned above are also included.

In one embodiment, the pharmaceutical composition comprises a neoplastic agent complexed with liposomes to stabilize and solubilize the neoplastic agent and allow its permeation into the bladder wall. Liposomes are phospholipid vesicles which have been designed as carrier systems for drugs, to procure either site specific pharmacological action or controlled release of the drug, thus enhancing efficacy while diminishing undesirable side effects. While not wishing to be limited by theory, liposomes could be appropriate vehicles for delivery of a neoplastic agent because (a) they would entrap and control release the neoplastic agent, (b) they would protect neoplastic agent from the biological environment, until it is released, (c) they provide a means of diminishing the toxicity of the neoplastic agent until it is released and (d) depending on the lipids used, they have the ability to target specific cells.

Liposomes can be prepared from many amphiphilic lipids and lipid mixtures, such as phospholipids, cholesterol, sphingolipids and fatty acid triglycerides. For example, suitable liposome formulations comprise combinations of phosphatidyl ethanolamine and phosphatidyl inositol with either cholesterol, oleic acid or diglyceride succinate. Further liposome formulations comprise combinations of phosphatidyl choline and cholesterol with either of the following sphingolipids: D-glucosyl-β1-1′ceramide (C8); D-glucosyl-β1-1′ceramide (C12); D-glucosyl-β1, N-palmitoyl-D-erythro-sphinosine; D-galactosyl-β1-1′ceramide (C8); D-galactosyl-β1-1′ceramide (12); D-galactosyl-β1-1′-N-Nervonyl-D-erythro-sphingosine; or D-glactose-β1-1′ ceramide (C8); D-glactose-β1-1′ ceramide (C12).

Upon hydration the phospholipid mixtures will organize into unilamella or multilamella bilayer structures. However, those mixtures containing phosphatidyl ethanol amine with either oleic acid or diglyceride succinate will organize into such structures at neutral pH. At acidic pH these structures will form nonbilayer structures which would enable membrane fusion. (Progress in Lipid Research 39 (2000) 409-460). The lamellar structures composed of the sphingolipids will contain a surface coat of carbohydrates that would be expected to interact strongly with and bind to the glycosaminoglycan or mucin layer of the bladder. The binding of these liposomes to the mucin layer will allow a targeted sustained release of vairubicin. While those phospholipids comprised of phosphatidyl ethanol amine, phosphatidyl inositol and either oleic acid or diglyceride succinate will bind to the mucin layer due to the pentahydroxycyclohexyl moiety of phosphatidyl inositol the release of valrubicin could be expected to be more rapid as the pH of the bladder decreases.

Treatment of a disease or condition may be accomplished in a subject by administration of neoplastic agent formulations as embodied herein. Administration of the compositions may be continuous or intermittent, depending, for example, upon the recipient's physiological condition, and other factors known to skilled practitioners. The administration of the formulations may be essentially continuous over a preselected period of time or may be in a series of spaced doses.

In some embodiments, the pharmaceutical compositions may be used in combination with one or more therapeutic agents for the treatment of cancer. In one embodiment, the pharmaceutical composition is combined with immunotherapy using Bacille Calmette-Guerin (BCG). BCG activates local type 1 (Th1) DTH-like immune responses which result in tumor necrosis.

In one embodiment, the neoplastic agent formulations are administered directly to a subject to achieve the desired response. The amount administered will vary depending on various factors including, but not limited to, the composition chosen, the particular disease, the weight, the physical condition, and the age of the subject, and whether prevention or treatment is to be achieved. Such factors can be readily determined by the clinician employing animal models or other test systems which are well known to the art.

Typically, an effective amount of the compositions sufficient for achieving a therapeutic or prophylactic effect, ranges from about 1 mg per intravesical administration to about 1,000 mg per intravesical administration. Preferably, the dosage ranges are from about 50 mg per intravesical administration to about 500 mg per intravesical administration.

An effective amount (e.g., dose) of neoplastic agent formulations described herein will provide therapeutic benefit without causing substantial toxicity to the subject. Toxicity of the neoplastic agent formulations described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in humans. The exact formulation, route of administration and dosage can be chosen by the individual physician in view of the subject's condition. See, e.g., Fingl et al., In: The Pharmacological Basis of Therapeutics, Ch. 1 (1975).

When the pharmaceutical compositions are prepared for administration, they are preferably combined with a pharmaceutically acceptable carrier, diluent or excipient to form a pharmaceutical formulation, or unit dosage form. The total active ingredients in such formulations include from 0.1 to 99.9% by weight of the formulation. The active ingredient for administration may be present as a powder or as granules; as a solution, a suspension or an emulsion.

Pharmaceutical formulations containing the neoplastic agents can be prepared by procedures known in the art using well known and readily available ingredients. The neoplastic agents can be formulated as solutions appropriate for parenteral administration, for instance by intramuscular, subcutaneous or intravenous routes. The pharmaceutical formulations of the neoplastic agents can also take the form of an aqueous or anhydrous solution or dispersion, or alternatively the form of an emulsion or suspension.

The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

The pharmaceutical formulations may include, as optional ingredients, pharmaceutically acceptable carriers, diluents, solubilizing or emulsifying agents, and salts of the type that are well-known in the art. Specific, non-limiting examples of the carriers and/or diluents that are useful in the pharmaceutical formulations include water and physiologically acceptable buffered saline solutions, such as phosphate buffered saline solutions pH 7.0-8.0.

Suitable carriers for parenteral solutions include water, suitable oil, saline, aqueous dextrose (glucose), related sugar solutions, and/or glycols such as propylene glycol or polyethylene glycols. Solutions for parenteral administration contain the active ingredient, suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium ethylenediaminetetraacetic acid (EDTA). In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

Additionally, standard pharmaceutical methods can be employed to control the duration of action. These are well known in the art and include control release preparations and can include appropriate macromolecules, for example polymers, polyesters, polyamino acids, polyvinyl, pyrrolidone, ethylenevinylacetate, methyl cellulose, carboxymethyl cellulose or protamine sulfate. The concentration of macromolecules as well as the methods of incorporation can be adjusted in order to control release. Additionally, the agent can be incorporated into particles of polymeric materials such as polyesters, polyamino acids, hydrogels, poly (lactic acid) or ethylenevinylacetate copolymers. In addition to being incorporated, these agents can also be used to trap the compound in microcapsules.

Accordingly, the pharmaceutical compositions may be delivered via various routes and to various sites in an mammal body to achieve a particular effect. One skilled in the art will recognize that although more than one route can be used for administration, a particular route can provide a more immediate and more effective reaction than another route. Local or systemic delivery can be accomplished by administration comprising application or instillation of the formulation into body cavities, inhalation or insufflation of an aerosol, or by parenteral introduction, comprising intramuscular, intravenous, peritoneal, subcutaneous, intradermal, as well as topical administration. In a preferred embodiment, the formulations of are provided to a subject intravesically, i.e., instilled into the bladder.

Examples of such carriers or diluents include, but are not limited to, water, saline, Ringer's solutions, dextrose solution, and 5% human serum albumin. As described above, liposomes and non-aqueous vehicles such as fixed oils may also be used. The use of such media and compounds for pharmaceutically active substances is well known in the art. Except insofar as any conventional media or compound is incompatible with the neoplastic agents, use thereof in the compositions is contemplated. Supplementary active compounds can also be incorporated into the compositions.

The present embodiments, thus generally described, will be understood more readily by reference to the following examples, which are provided by way of illustration and are not intended to be limiting of the present technology in any way.

### EXAMPLES

The following Tables further illustrate various embodiments, and should not be construed as limiting in any way. The tables are listings of vairubicin formulations.

TABLE 1
Valrubicin Formulations Containing DMSO

| | Formulation | | | | | | |
|---|---|---|---|---|---|---|---|
| Compound | Valstar ® | 1 | 2 | 3 | 4 | 5 | 6 |
| Valrubicin | 40 | 40 | 40 | 40 | 40 | 40 | 40 |
| (mg) | | | | | | | |
| Ethanol | 0.50 | | | | | | |
| (mL) | | | | | | | |
| DMSO (mL) | | 0.50 | 0.25 | 0.25 | 0.25 | 0.25 | |
| Cremophor | 0.50 | 0.50 | | | | | |
| EL (mL) | | | | | | | |
| Polysorbate | | | 0.75 | 0.75 | 0.75 | 0.75 | 0.50 |
| 80 (mL) | | | | | | | |
| Polyacrylic | | | | 28 | | | |
| Acid (mg) | | | | | | | |
| Polyvinyl | | | | | 113 | | |
| pyrrolidone | | | | | | | |
| K-17 (mg) | | | | | | | |
| Sodium | | | | | | 14 | |
| Alginate | | | | | | | |
| (mg) | | | | | | | |
| Polyethylene | | | | | | | 0.50 |
| Glycol 400 | | | | | | | |
| (mL) | | | | | | | |
| Poloxamer | | | | | | | 525 |
| 407 (mg) | | | | | | | |
| Saline (mL) | | | | 2.75 | 2.75 | 2.75 | 2.75 |

TABLE 2
Selected Lipid Formulations

| | | | PC^{(b)} | Lyso-PC | DOTAP | Glycolipid | Valrub. |
|---|---|---|---|---|---|---|---|
| Form. # | Ratio | AO^{(a)} | (mg/mL) | (mg/ml)^{(c)} | (mg/ml)^{(d)} | (mg/ml)^{(e)} | (mg/mL) |
| 7 | DOPC/Lyso-oleoyl-PC | No | 79.7 | 6.44 | | | 10.0 |
| | (9/1 mole ratio) | | | | | | |
| 8 | Soy PC/Lyso-soy-PC | Yes | 67.3 | 22.1 | | | 12.1 |
| | (7/3 mole ratio) | | | | | | |
| 9 | Soy PC/Lyso-Soy- | Yes | 55.4 | 22.6 | 9.36 | | 11.2 |
| | PC/DOTAP (6/3/1 | | | | | | |
| | mole ratio) | | | | | | |
| 10 | Soy PC/lyso-Soy-PC/ | No | 58.0 | 21.9 | 9.50 | | 11.2 |
| | DOTAP (6/3/1 mole | | | | | | |
| | ratio) | | | | | | |
| 11 | Soy PC/lyso-Soy-PC/ | Yes | 69.5 | 20.9 | | N/D | 11.7 |
| | Glycolipid A (69/30/1 | | | | | | |
| | mole ratio) | | | | | | |
| 12 | Soy PC/lyso-Soy-PC/ | Yes | 70.0 | 21.0 | | N/D | 11.5 |
| | Glycolipid B (69/30/1 | | | | | | |
| | mole ratio) | | | | | | |
| 13 | Soy PC/lyso-Soy-PC/ | Yes | 59.5 | 21.0 | 9.41 | N/D | 11.2 |
| | DOTAP/Glycolipid A | | | | | | |
| | (59/30/10/1 mole ratio) | | | | | | |

^{(a)}Antioxidants are Tocopherol and Ascorbate-6-palmitate at 0.1 wt % each to total lipid.
^{(b)}PC = phosphatidylcholine DOPC = dioleoylphosphatidylcholine Soy PC = Soy Phosphatidylcholine
^{(c)}Lyso-PC = 1-Acyl-2-Hydroxy-sn-Glycero-3-Phosphocholine
^{(d)}DOTAP = 1,2-Diacyl-3-Dimethylammonium-Propane (DAP)
^{(e)}Glycolipid—Glycolipid A = D-Glucosyl-β1-1′-N-Dodecanoyl-D-erythro-Sphingosine (C12 β-D-Glucosyl Ceramide); Glycolipid B = D-Lactosyl-β1-1′-N-Dodecanoyl-D-erythro-Sphingosine (C12 β-D-Lactosyl Ceramide)
N/D indicates that the amount of glycolipid was not determined in terms of mg/ml.

### Example 1

In this example, various formulations identified in the tables above and below were instilled in the bladders of rats. The rats were then sacrificed at a predetermined interval and blood and bladders were collected. The blood was analyzed for systemic penetration of the valrubicin. The bladders were analyzed for inflammation by scoring each bladder on five parameters: venous congestion, edema, epithelial damage, hemorrhage, and cellular infiltration, scored on a scale from zero to ten, where numbers in between describe varying degrees of the parameters measured. For edema, a zero corresponds to no edema, while a ten corresponds to dramatic focal edema involving the entire bladder. For venous congestion, a zero corresponds to no venous congestion, while a ten corresponds to all visible venous vessels being significantly dilated. For cellular infiltration, a zero corresponds to no cellular infiltration, while a ten corresponds to very severe cellular infiltration suggesting infection (presence of neutrophils). For epithelial damage, a zero corresponds to no epithelial damage, while a ten corresponds to significant loss of major areas of epithelia. For hemorrhage, a zero corresponds to no hemorrhage, while a ten corresponds to all profound extensive hemorrhage. The five individual scores are then summed to provide a total inflammation score for each animal. Then the number of animals used for any particular formulation was included to determine the average inflammatory score for that formulation. Lower inflammation scores are to be believed to be associated with lower amounts of irritation of the bladder.

TABLE 3
Inflammation/irritation Test Results

| | | | Mean for Each | | | | | | | |
|---|---|---|---|---|---|---|---|---|---|---|
| | | | Parameter Based on | | | | | Total Inflammation | | |
| | | | the # of Animals | | | | | Score | | |
| Form. #¹ | Sal. Dil.² | Animals Tested | VC | E | CI | ED | H | Mean | Std. Dev. | SEM |
| Saline | None | 7 | 1.9 | 4.0 | 2.0 | 2.0 | 0 | 9.9 | 4.7 | 1.9 |
| Control | | | | | | | | | | |
| Valstar ® | 1:1 | 7 | 8.0 | 8.6 | 8.7 | 8.0 | 6.7 | 40 | 12.8 | 5.7 |
| Valstar ® | 1:2.75 | 6⁵ | 4.7 | 6.2 | 5.7 | 4.0 | 1.5 | 22.0 | 6.7 | 7.2 |
| 1 | 1:1 | 6 | 2.2 | 3.7 | 2.7 | 1.8 | 0.2 | 10.5 | 5.8 | 2.6 |
| 1 | 1:2.75 | 6⁶ | 2.7 | 5.0 | 2.0 | 1.3 | 0.5 | 11.5 | 3.7 | 1.6 |
| 4 | None | 7 | 4.6 | 5.7 | 4.6 | 2.6 | 1.7 | 20.3 | 10.1 | 4.5 |
| 8 | None | 5 | 3.2 | 7.4 | 5.4 | 3.4 | 0.0 | 19.4 | 5.1 | 2.3 |
| 9 | None | 5 | 4.6 | 4.8 | 3.4 | 3.2 | 0.0 | 16.0 | 10.8 | 4.8 |
| 11 | None | 4⁷ | 6.0 | 6.5 | 3.3 | 2.8 | 0.8 | 19.0 | 8.3 | 4.1 |
| 12 | None | 3⁸ | 6.9 | 7.4 | 4.4 | 2.9 | 1.0 | 23.2 | 20.2 | 11.7 |

¹See Tables 1 and 2 for formulation contents.
²Sal. Dil. refers to a saline dilution of the formulation with a saline solution on a volume to volume basis, e.g. formulation volume:saline volume.
³The mean inflammation score is the mean of the total inflammation score for each animal tested. Std. Dev. is an abbreviation for stand deviation. SEM is an abbreviation for standard error of the mean.
⁴Parameter Abbreviations: VC refers to venous congestion; E refers to edema; CI refers to cellular infiltration; ED refers to epithelial damage; and H refers to hemorrhage.
⁵Seven animals were to be tested, but had an infection and the results were excluded.
⁶Seven animals were to be tested, but one died during testing and the bladder was not tested.
⁷Seven animals were to be tested, but one died during testing and the bladder was not tested. The animal was about 20 g smaller than those in the control, and Formulations 4, 8, and 9, thus, the anesthetic used during instillation may have been too substantial.
⁸Seven animals were to be tested, but two died during testing and the bladders were not tested. The animals were about 20 g smaller than those in the control, and Formulations 4, 8, and 9, thus, the anesthetic used during instillation may have been too substantial.

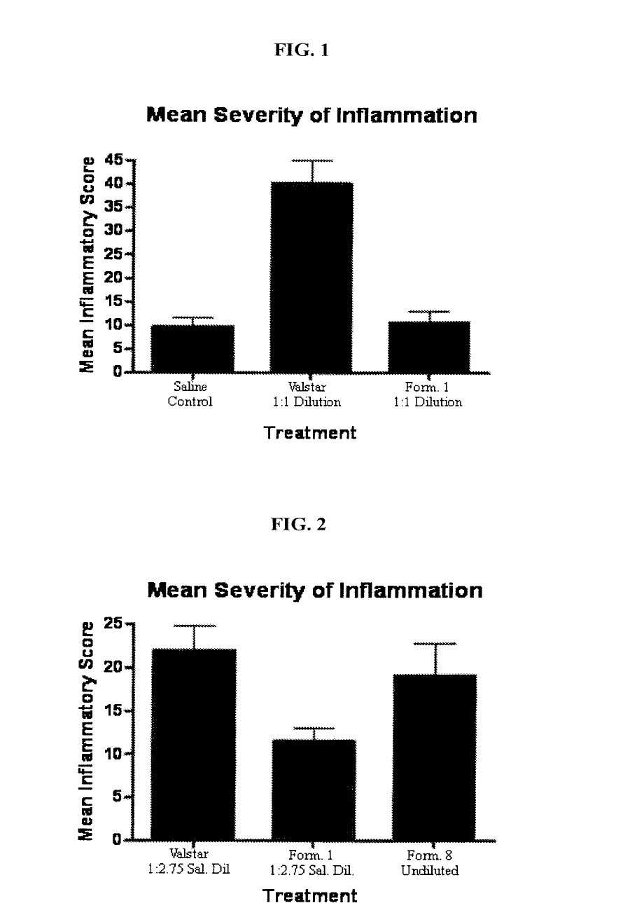

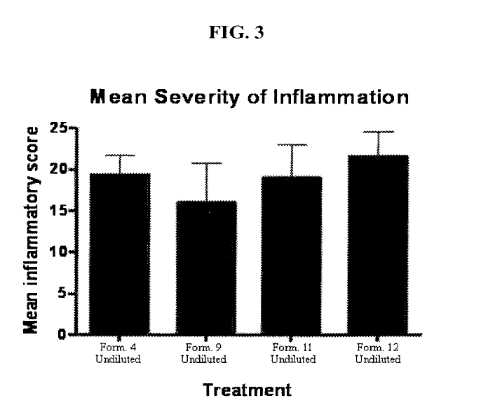

FIGS. 1-3 illustrate graphically the results presented in Table 3. FIG. 1 illustrates graphically the inflammation of rat (animal) bladders as a result of instillation of the noted formulation. A simple saline solution results in an average inflammation score of approximately 10. A standard Valstar® formulation, having 1:1 dilution with saline, results in a significantly higher inflammation score of approximately 40. The instillation of Formulation 1, at a 1:1 saline dilution, results in an inflammation score approximately equal to that of the saline instillation. Hence, Formulation 1 is significantly less irritating to the bladder than the present standard commercial formulation of valrubicin. FIG. 2 illustrates graphically the inflammation of rat (animal) bladders as a result of instillation of Valstar® at a 1:2.75 saline dilution in comparison to Formulations 1 (1:2.75 dilution) and 8 (undiluted). While Formulation 1 had significantly less (ρ=0.007) irritation than the standard Valstar® formulation, Formulation 8, although less than the standard formulation, was not statistically significantly different with regard to inflammation from the standard Valstar® formulation. FIG. 3 illustrates a comparison of Formulations 4, 9, 11, and 12. While the absolute values seems to vary from sample to sample, the differences do not appear to be statistically significant. In FIGS. 2 and 3, the valrubicin concentration was approximately the same in all of the solutions instilled into the bladder. For example, Valstar® and Formulation 1 at 1:2.75, and undiluted Formulations 4, 8, 9, 11, and 12, all had a theoretical valrubicin concentration of approximately 11 mg/mL.

The present disclosure is not to be limited in terms of the particular embodiments described in this application. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A pharmaceutical composition comprising an effective amount of valrubicin and dimethyl sulfoxide in an intravesical dosage form.

2. The pharmaceutical composition of claim 1, wherein the effective amount of valrubicin is from about 5 mg/mL to about 100 mg/mL, from about 10 mg/mL to about 90 mg/mL, from about 15 mg/mL to about 80 mg/mL, from about 20 mg/mL to about 70 mg/mL, from about 25 mg/mL to about 70 mg/mL, from about 30 mg/mL to about 60 mg/mL, from about 35 mg/mL to about 50 mg/mL, or from about 35 mg/mL to about 45 mg/mL.

3. The pharmaceutical composition of claim 1 comprising one or more additional chemical permeation enhancers selected from the group consisting of: ethanol, isopropanol, dimethylacetamide, dimethylformamide, decylmethylsulfoxide, 2-pyrrolidone, N-ethyl-2-pyrrolidone, capric acid, linoleic acid, ureas, sodium dodecyl sulfate, sodium lauryl sulfate, and mixtures of any two or more thereof.

4. The pharmaceutical composition of claim 1, wherein the effective amount of valrubicin and dimethyl sulfoxide is sufficient to treat bladder cancer.

5. The pharmaceutical composition of claim 1 comprising a junction opener.

6. The pharmaceutical composition of claim 5, wherein the junction opener is selected from the group consisting of: trimethyl-chitosan, mono-N-carboxymethyl chitosan, N-diethyl methyl chitosan, sodium caprate, cytochalasin B, IL-1, polycarbophil, carbopol 934P, N-sulfato-N,O-carboxymethylchitosan, Zounla occludens toxin, 1-palmitoyl-2-glutaroyl-sn-glycero-3-phosphocholine, and mixtures of any two or more thereof.

7. The pharmaceutical composition of claim 5, wherein the amount of the junction opener is from about 1 to about 15 percent by weight/volume of the dosage form.

8. The pharmaceutical composition of claim 1 comprising a polyethoxylated castor oil.

9. The pharmaceutical composition of claim 8, wherein the polyethoxylated castor oil is Cremophor.

10. The pharmaceutical composition of claim 9, wherein the Cremophor and dimethyl sulfoxide are provided in equal amounts.

11. The pharmaceutical composition of claim 1 further comprising a mucin-degrading compound.

12. The pharmaceutical composition of claim 11, wherein the mucin-degrading compound is selected from the group consisting of: trypsin, hyaluronidase, protamine sulfate, and norepinephrine.

13. The pharmaceutical composition of claim 1 further comprising a bioadhesive or mucoadhesive agent.

14. The pharmaceutical composition of claim 13, wherein the mucoadhesive agent is polyacrylic acid.

15. The pharmaceutical composition of claim 1 further comprising an ionic or non-ionic surfactant, a polyvinyl pyrrolidone, alginates, a polyacrylic acid, or a mixture of any two or more thereof.

16. The pharmaceutical composition of claim 15, wherein the ionic and non-ionic surfactants are polyoxyethylene castor oil derivatives, block copolymers of ethylene oxide and propylene oxide, sorbitan fatty acid esters, or a mixture of any two or more thereof.

17. The pharmaceutical composition of claim 16, wherein the polyacrylic acids are Carbomer 934P, Carbomer 940, Carbomer 941, Carbomer 974P, Carbomer 980, Carbomer 1342, polycarbophil, calcium polycarbophil, or a mixture of any two or more thereof.

18. A pharmaceutical composition comprising an effective amount of valrubicin and 2-hydroxy-propyl-β-cyclodextran in an intravesical dosage form.

19. A pharmaceutical composition comprising:
a liposomal dosage form comprising an effective amount of liposome-entrapped valrubicin;
wherein, the liposome comprises at least one liposome forming material selected from the group consisting of: phosphatidyl choline and phosphatidyl ethanolamine.

20. A method for treating bladder cancer comprising administering the pharmaceutical composition of claim 1.

21. A method for treating bladder cancer comprising administering the pharmaceutical composition of claim 18.

22. A method for treating bladder cancer comprising administering the pharmaceutical composition of claim 19.
